(19) United States
(12) Patent Application Publication
(10) Pub. No.: US 2026/0301161 A1
(43) Pub. Date: Oct. 1, 2026

(54) MEDICAL IMAGE PROCESSING APPARATUS AND MEDICAL IMAGE PROCESSING METHOD

(71) Applicant: Canon Kabushiki Kaisha, Tokyo (JP)

(72) Inventors: Christophe MEYERS, Edinburgh (GB); Itsuki KUGA, Utsunomiya (JP); Morvyn MYLES, Edinburgh (GB); Magnus Fredrik WAHRENBERG, Edinburgh (GB)

(73) Assignee: Canon Kabushiki Kaisha, Tokyo (JP)

(21) Appl. No.: 19/093,455

(22) Filed: Mar. 28, 2025

(51) Int. Cl. G06T 7/00; G06T 15/60

(52) U.S. Cl. CPC G06T 7/0012; G06T 15/60; G06T 2207/10132

(57) ABSTRACT

A medical image processing apparatus comprising processing circuitry configured to acquire three-dimensional image data of an anatomical region of a subject, generate first image data by rendering the three-dimensional image data using a first rendering method, generate second image data by rendering the three-dimensional image data using a second rendering method and depth information and generate combined image data representing the anatomical region of the subject based on the first image data and the second image data.

### FIELD

The present disclosure relates to a medical image processing apparatus, a medical image processing method and a medical imaging system.

### BACKGROUND

Ultrasound images are formed by sending pulses of high frequency sound waves into tissue from an ultrasound probe. These pulses echo off tissues within a patient with different reflection properties and are returned to and detected at the probe. The ultrasound scanner uses the measurement of these reflected pulses to construct an image. Three-dimensional (3D) ultrasound data can be obtained by a specifically designed probe for collecting the 3D data. Alternatively, 3D ultrasounds data may be obtained by collecting a plurality of ultrasound images obtained by moving an ultrasound probe. For example, the ultrasound probe may be tilted, with reflected pulses being captured at different orientations of the probe. These reflected pulses captured at different orientations of the probe are processed to produce a three-dimensional array comprising a plurality of voxels representing the imaged structure. After capturing the 3D data, a two-dimensional (2D) image is produced from a selected angle by applying a volume rendering technique to the 3D data. The 2D image that is produced may comprise a helpful visualisation of the raw information captured by the ultrasound scanner.

When rendering the 3D data, e.g. using a transparent rendering method, it may be difficult to determine from the produced 2D image which features of the anatomical region or structure are in the foreground and which features are in the background. The produced 2D image may be superimposed with another 2D image, which has been obtained using another rendering method, e.g. to provide additional context. For example, the 3D data may be rendered using a first rendering method for visualising a surface of an anatomical objection, such as a foetus. The 3D data may be additionally rendered using a second rendering method for visualising an internal structure of the anatomical object. The 2D data that is produced by each of the first and second rendering methods may be blended using a blending method. However, using a simple blending method may lead to artefacts and/or noise in the blended 2D data.

### DESCRIPTION

Embodiments are now described by way of non-limiting example with reference to the accompanying drawings in which:

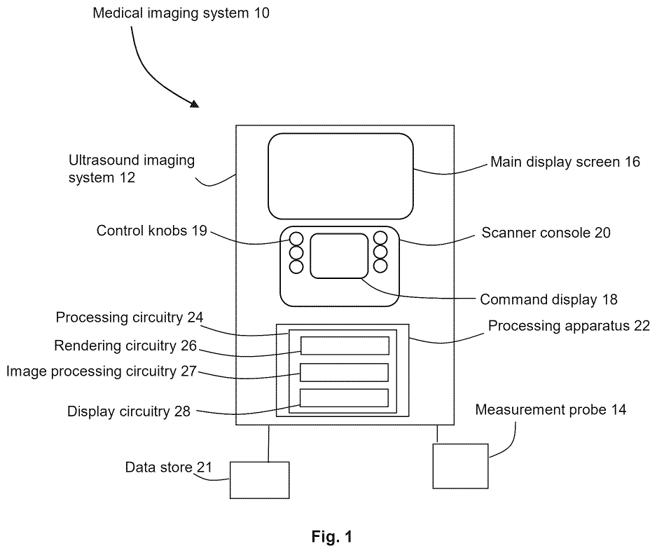

FIG. 1 is a schematic illustration of a medical imaging system according to an embodiment;

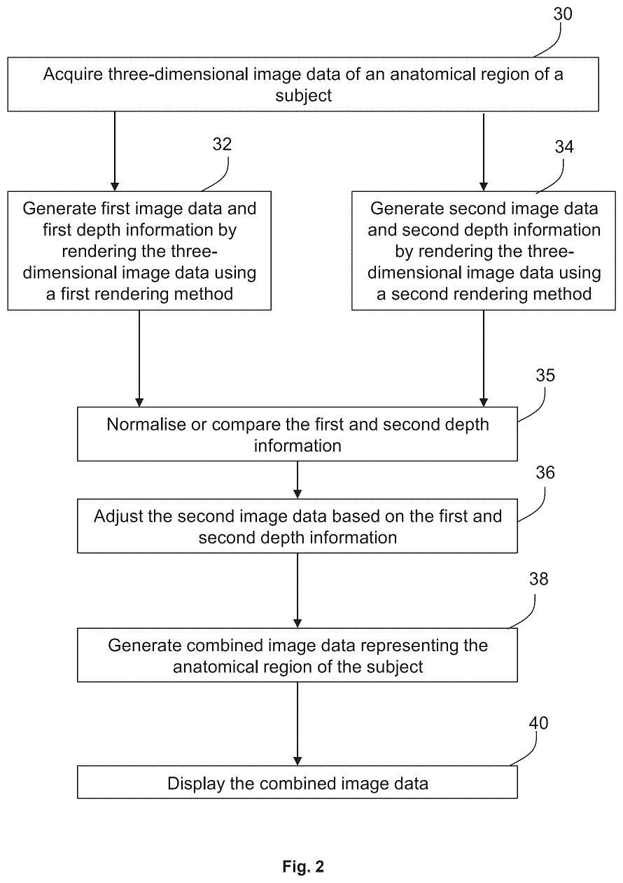

FIG. 2 is a flow chart illustrating in overview a process according to an embodiment;

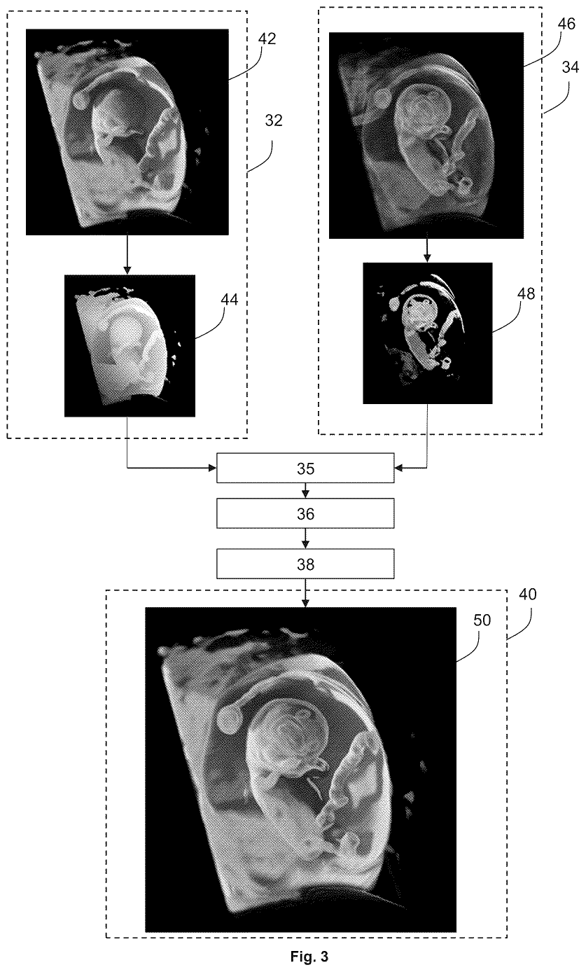

FIG. 3 is another flow chart illustrating in overview the process of FIG. 2;

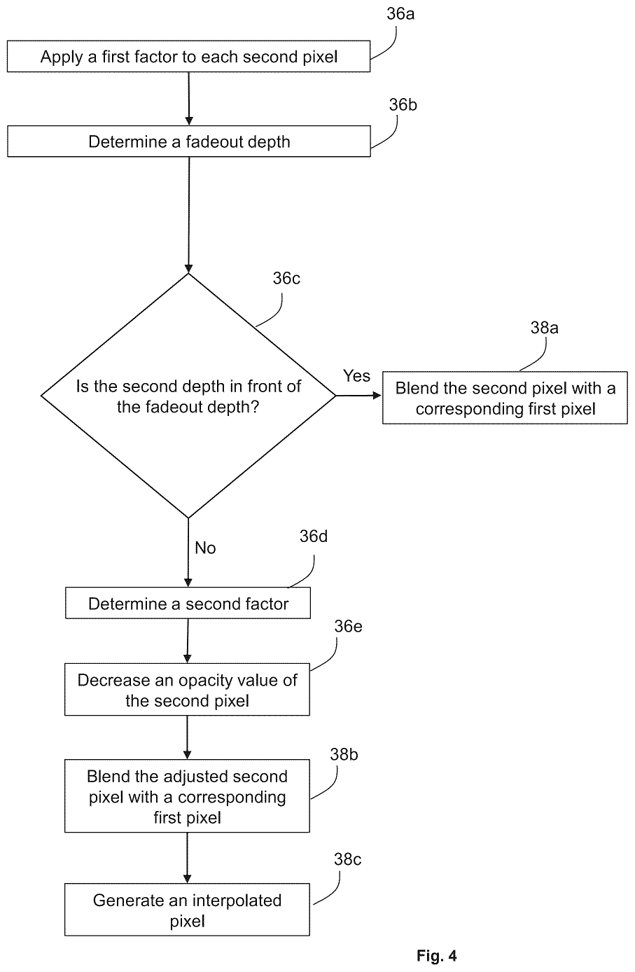

FIG. 4 is a flow chart illustrating in overview further stages of the process of FIGS. 2 and 3;

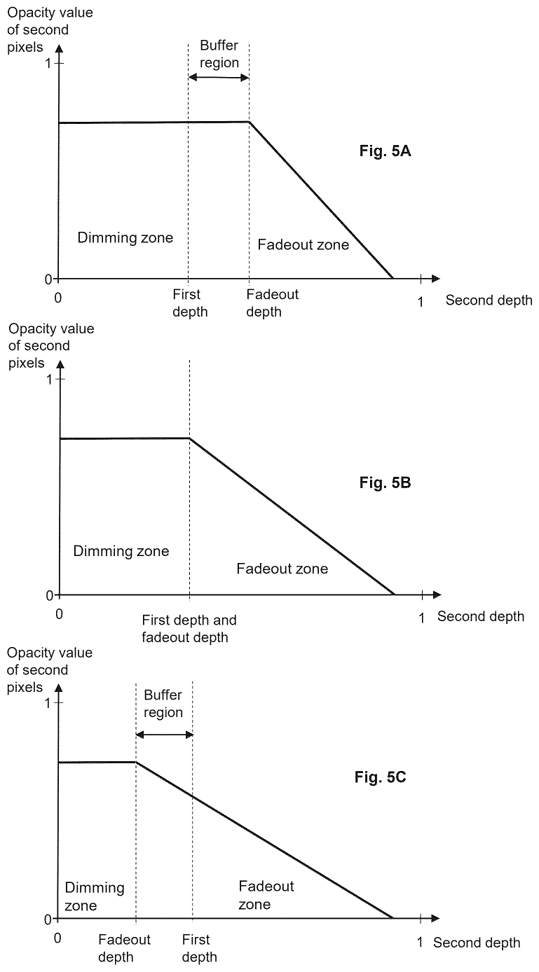

FIG. 5A illustrates an exemplary graph of an opacity value of second pixels in dependence on a second depth;

FIG. 5B illustrates another exemplary graph of an opacity value of second pixels in dependence on a second depth;

FIG. 5C illustrates another exemplary graph of an opacity value of second pixels in dependence on a second depth;

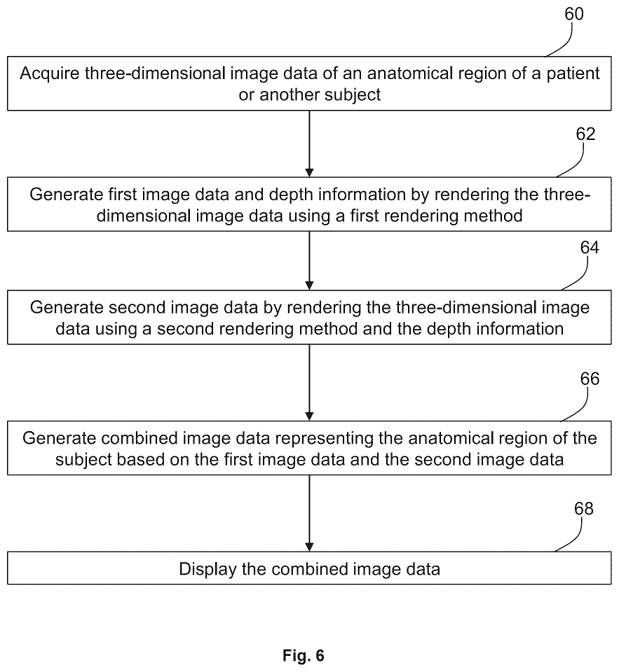

FIG. 6 is a flow chart illustrating in overview a process according to another embodiment;

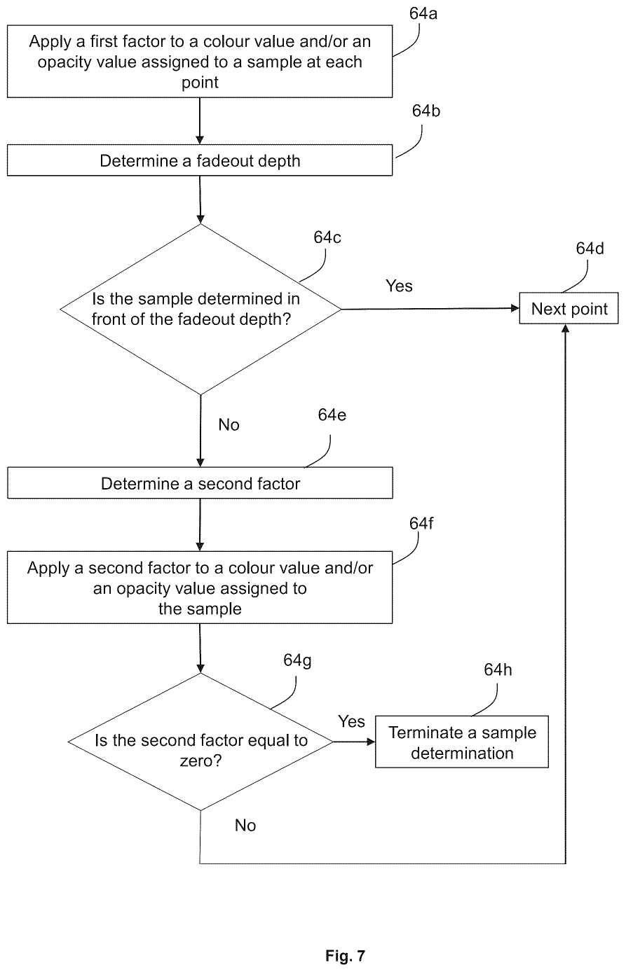

FIG. 7 is a flow chart illustrating in overview further stages of the process of FIG. 6;

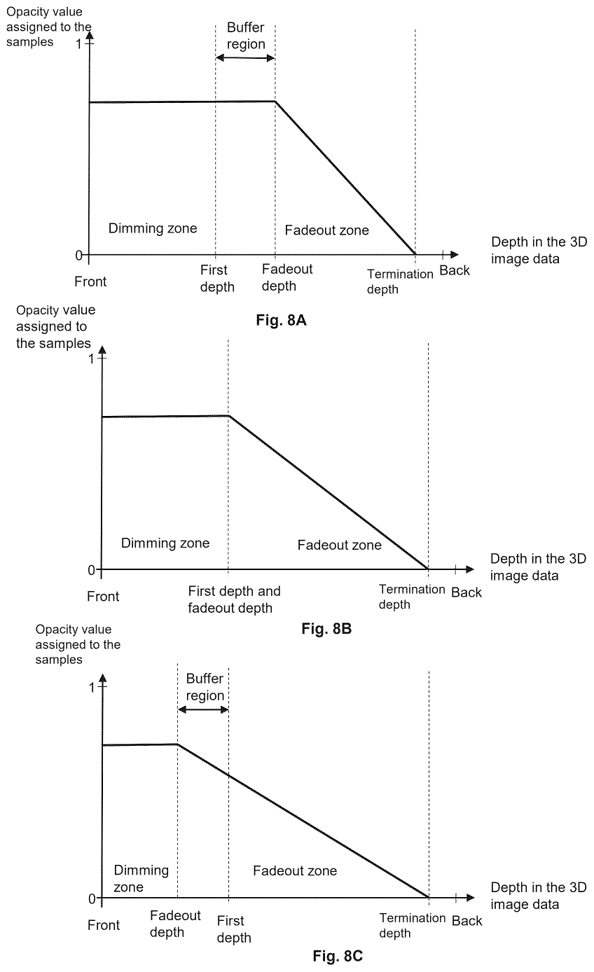

FIG. 8A illustrates an exemplary graph of an opacity value assigned to a number of samples in dependence on a depth in 3D image data;

FIG. 8B illustrates another exemplary graph of an opacity value assigned to a number of samples in dependence on a depth in 3D image data;

FIG. 8C illustrates another exemplary graph of an opacity value assigned to a number of samples in dependence on a depth in 3D image data;

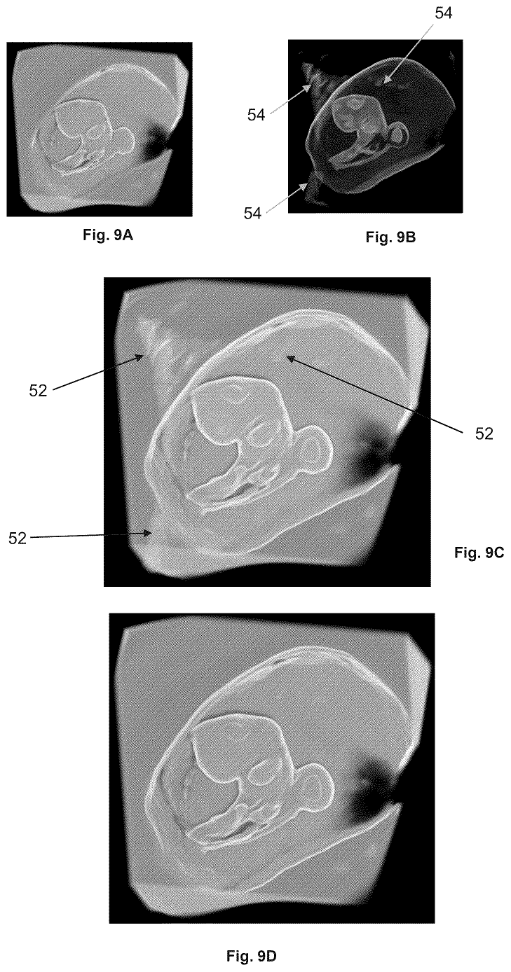

FIG. 9A illustrates a 2D image representing first image data;

FIG. 9B illustrates a 2D image representing second image data;

FIG. 9C illustrates a 2D image representing a blend of the first and second image data of FIGS. 9A and 9B respectively;

FIG. 9D illustrates a 2D image that has been generated using the process of FIGS. 2 and 3;

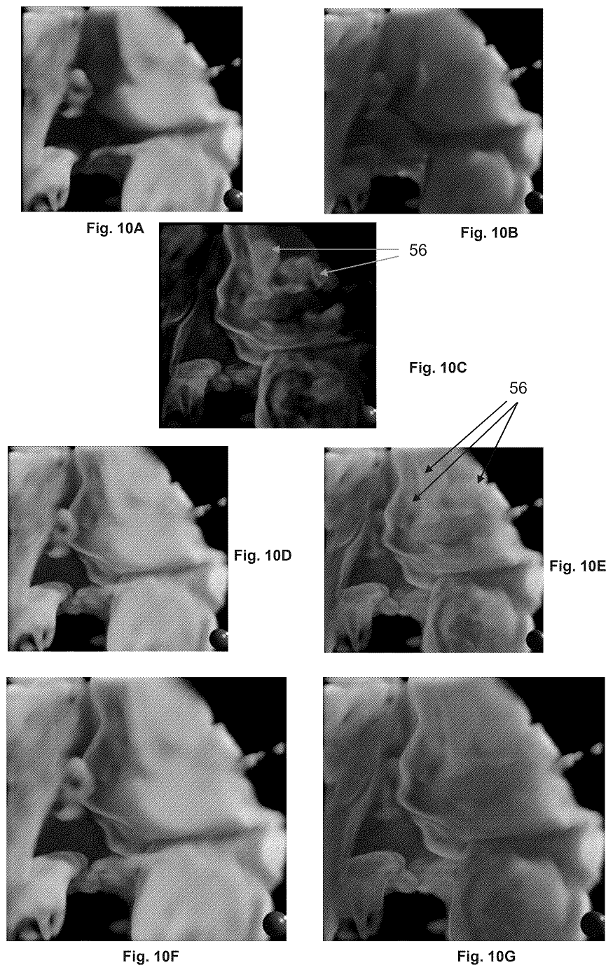

FIG. 10A illustrates another exemplary 2D image representing first image data;

FIG. 10B illustrates another exemplary 2D image representing first image data;

FIG. 10C illustrates another exemplary 2D image representing second image data;

FIG. 10D illustrates a 2D image representing a blend of the first and second image data of FIGS. 10A and 10C;

FIG. 10E illustrates a 2D image representing a blend of the first and second image data of FIGS. 10B and 100;

FIG. 10F illustrates a 2D image that has been generated using the process of FIGS. 2 and 3;

FIG. 10G illustrates a 2D image that has been generated using the process of FIGS. 2 and 3; and

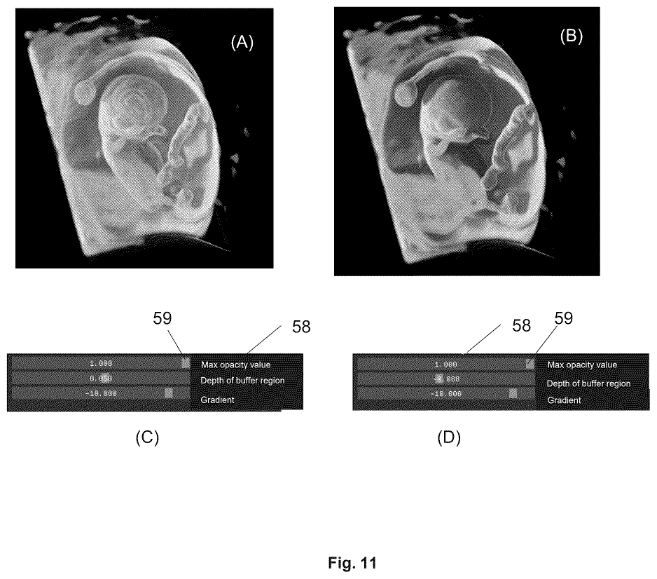

FIG. 11 illustrates a comparison of two 2D images that have been generated using the process of FIGS. 2 and 3.

Certain embodiments provide a medical image processing apparatus comprising processing circuitry configured to acquire three-dimensional image data of an anatomical region of a subject, generate first image data by rendering the three-dimensional image data using a first rendering method, generate second image data by rendering the three-dimensional image data using a second rendering method and depth information and generate combined image data representing the anatomical region of the subject based on the first image data and the second image data.

The subject may comprise a patient.

The depth information may be generated using the first rendering method. For example, the processing circuitry may be configured to generate the depth information by rendering the three-dimensional image data using the first rendering method.

When using the second rendering method, the processing circuitry may be configured to determine, e.g. successively determine, a plurality of samples. The plurality of samples may correspond to a plurality of points in the three-dimensional image data. When using the second rendering method, the processing circuitry may be configured to assign at least one of: a colour value and an opacity value to each of the plurality of samples. When using the second rendering method, the processing circuitry may be configured to apply a first factor to adjust at least one of: the colour value and the opacity value assigned to each of the plurality of samples. The processing circuitry may be configured to apply the first factor to adjust at least one of: the colour value and opacity value assigned to each of the plurality of samples. For example, the processing circuitry may be configured to apply the first factor to adjust a maximum value of at least one of: the colour value and opacity value.

The processing circuitry may be configured to determine at each of the plurality of points whether a respective sample is determined in front of a designated depth. The designated depth may be designated or determined in a viewing direction of the first rendering method.

The processing circuitry may be configured to maintain at least one of the colour value and opacity value assigned to a sample that is determined in front of the designated depth as constant or unchanged.

In response to a determination that the sample is determined in front of the designated depth, the processing circuitry may be configured to determine whether a sample at a subsequent point of the plurality of points is determined in front of the designated depth.

The processing circuitry may be configured to change, e.g. gradually change, at least one of the colour value and opacity value assigned to a sample that is determined behind or not in front of the designated depth.

At a termination depth, the processing circuitry may be configured to terminate the second rendering method.

For example, in response to a determination that the sample is not determined in front of the designated depth, the processing circuitry may be configured to determine a second factor. The second factor may be associated with the sample.

The processing circuitry may be configured to apply the second factor to at least one of: the colour value and the opacity value assigned to the sample. The processing circuitry may be configured to determine whether the second factor is equal to zero. In response to a determination that the second factor is not equal to zero, e.g. larger than zero, the processing circuitry may be configured to determine whether a sample at a subsequent point of the plurality of points is determined in front of the designated depth. Alternatively, when the second factor is equal to zero, the processing circuitry may be configured to terminate a determination of the plurality of samples. The second factor may be equal to zero at the termination depth.

The processing circuitry may be configured to determine or designate a designated depth, e.g. based on at least one of: the depth information and a depth of a buffer region.

For example, the processing circuitry may be configured to determine or designate the designated depth based on a surface depth of the anatomical region. The surface depth may be identified by rendering the three-dimensional image data using the first rendering method. The surface depth may be part of or comprised in the depth information.

The processing circuitry may be configured to determine or designate the designated depth based on the depth of the buffer region and the surface depth of the anatomical region. The designated depth may be adjustable.

The first rendering method may comprise a rendering method for visualising a surface of the anatomical region of the subject. The second rendering method may comprise a method for visualising an internal structure of the anatomical region of the subject. The first rendering method may comprise at least one of a surface rendering method and a global illumination rendering method, e.g. a first global illumination rendering method. The second rendering method may comprise at least one of a global illumination rendering method, e.g. a second global illumination method, and a surface rendering method. The second rendering method may comprise a transparent rendering method or a ShadowGlass method.

The processing circuitry may be configured to determine the depth of the buffer region, e.g. based on one or more properties of the anatomical region. The processing circuitry may be configured to automatically determine the depth of the buffer region, e.g. based on the one or more properties of the anatomical region.

The processing circuitry may be configured to maintain at least one of: the colour value and the opacity value assigned to each of the plurality of samples as constant or unchanged in the buffer region. Alternatively, the processing circuitry may be configured to vary at least one of: the colour value and the opacity value assigned to each of the plurality of samples in the buffer region, e.g. based on or according to a second factor. For example, when the depth of the buffer region is negative, the processing circuitry may be configured to start varying the at least one of: the colour value and the opacity value assigned to each of the plurality of samples in the buffer region, e.g. based on or according to a second factor.

Certain embodiments provide a medical image processing method comprising acquiring three-dimensional image data of an anatomical region of a subject, generating first image data by rendering the three-dimensional image data using a first rendering method, generating second image data by rendering the three-dimensional image data using a second rendering method and depth information and generating combined image data representing the anatomical region of the subject based on the first image data and the second image data.

Certain embodiments provide a medical imaging system comprising a medical image processing apparatus, the medical image processing apparatus comprising processing circuitry configured to acquire three-dimensional image data of an anatomical region of a subject, generate first image data by rendering the three-dimensional image data using a first rendering method, generate second image data by rendering the three-dimensional image data using a second rendering method and depth information and generate combined image data representing the anatomical region of the subject based on the first image data and the second image data.

Certain embodiments provide a medical image processing apparatus comprising processing circuitry configured to acquire three-dimensional image data of an anatomical region of a subject, generate first image data by rendering the three-dimensional image data using a first rendering method, generate second image data by rendering the three-dimensional image data using a second rendering method, adjust the second image data based on depth information and generate combined image data based on the first image data and the adjusted second image data.

The depth information may comprise first depth information. The depth information may comprise second depth information. The processing circuitry may be configured to generate the first depth information by rendering the three-dimensional image data using the first rendering method. The processing circuitry may be configured to generate the second depth information by rendering the three-dimensional image data using the second rendering method.

The second image data may comprise a plurality of second pixels.

The processing circuitry may be configured to apply a first factor to each of the plurality of second pixels, e.g. to adjust a value, e.g. a maximum value, of at least one channel of each of the plurality of second pixels.

The processing circuitry may be configured to determine for each of the plurality of second pixels whether a second depth associated with a second pixel is in front of a designated depth designated or determined in a viewing direction of the first rendering method.

The processing circuitry may be configured to maintain a value of at least one channel of a second pixel associated with a second depth in front of the designated depth as constant or unchanged. For example, in response to a determination that the second depth associated with the second pixel is in front of the designated depth, the processing circuitry may be configured to maintain the value of the at least one channel of the second pixel as constant or unchanged. In response to the determination that the second depth associated with the second pixel is in front of the designated depth, the processing circuitry may be configured to blend the second pixel with a corresponding first pixel, e.g. using a blend mode.

The blend mode may comprise at least one of a lighten blend mode, a darken blend mode, a multiply blend mode, an addition blend mode, an in blending mode, an atop blending, an overlay blend mode or any other suitable blend mode.

The processing circuitry may be configured to change, e.g. gradually change, a value of at least one channel of a second pixel associated with a second depth behind or not in front of the designated depth. This may generate an adjusted second pixel. For example, in response to the determination that the second depth associated with the second pixel is not in front the designated depth, the processing circuitry may be configured to determine a second factor for changing the value of at least one channel of the second pixel. The second factor may be determined based on one or more of: the depth of the buffer region, the first and second depth information and a gradient. The second factor may be associated with the second pixel. The processing circuitry may be configured to change the value of at least one channel of the second pixel based on or according to the second factor, e.g. to generate the adjusted second pixel. For example, the processing circuitry may be configured to apply the second factor to value of the at least one channel of the second pixel, e.g. to generate the adjusted second pixel.

The gradient may comprise a linear gradient or a non-linear gradient. The gradient may be an input parameter. The input parameter may be adjustable and/or specified by a user. The processing circuitry may be configured to decrease the value of at least one channel of the second pixel based on or according to the second factor.

The processing circuitry may be configured to blend the adjusted second pixel with a corresponding first pixel, e.g. using a blend mode to generate a blended pixel. The processing circuitry may be configured to generate an interpolated pixel based on the blended pixel and the corresponding first pixel. The combined image data may comprise the interpolated pixel. The processing circuitry may be configured to apply the second factor as an interpolation factor to the blended pixel, e.g. to generate the interpolated pixel.

The processing circuitry may be configured to determine or designate a designated depth based on at least one of the first depth information and a depth of a buffer region.

For example, the processing circuitry may be configured to determine or designate the designated depth based on a surface depth of the anatomical region. The surface depth may be identified by rendering the three-dimensional image data using the first rendering method. The surface depth may be part of or comprised in the first depth information.

The processing circuitry may be configured to determine or designate the designated depth based on the depth of the buffer region and the surface depth of the anatomical region. The designated depth may be adjustable.

The processing circuitry may be configured to determine the depth of the buffer region, e.g. based on one or more properties of the anatomical region. The processing circuitry may be configured to automatically determine the depth of the buffer region based on the one or more properties of the anatomical region.

The processing circuitry may be configured to maintain the value of at least one channel of at least one or each second pixel of the plurality of second pixels as constant or unchanged in the buffer region. Alternatively, the processing circuitry may be configured to vary the value of at least one channel of the at least one or each second pixel of the plurality of second pixels in the buffer region, e.g. based on or according to the second factor. For example, when the depth of the buffer region is negative, the processing circuitry may be configured to start varying the value of the at least one or each second pixel of the plurality of second pixels in the buffer region, e.g. based on or according to a second factor.

In a first iteration, the processing circuitry may be configured to render the three-dimensional image data using the first rendering method and to render the three-dimensional image data using the second rendering method.

In one or more subsequent iterations, the processing circuitry may be configured to alternately render the three-dimensional image using at least one of: the first and second rendering methods, e.g. to generate at least one of: updated first depth information and updated second depth information and at least one of: updated first image data and updated second image data, respectively.

The processing circuitry may be configured to adjust the updated second image data based on previously generated first depth information and the updated second depth information. Alternatively, the processing circuitry may be configured to adjust previously generated second image data based on the updated first depth information and the previously generated second depth information.

The processing circuitry may be configured to generate the combined image data based on at least one of: the previously generated first image data and the adjusted previously generated second image data and at least one other of: the updated first image data and the adjusted updated second image data.

Certain embodiments provide a medical image processing method comprising acquiring three-dimensional image data of an anatomical region of a subject, generating first image data by rendering the three-dimensional image data using a first rendering method, generating second image data by rendering the three-dimensional image data using a second rendering method, adjusting the second image data based on depth information and generating combined image data based on the first image data and the adjusted second image data.

Certain embodiments provide a medical imaging system comprising a medical image processing apparatus comprising processing circuitry configured to acquire three-dimensional image data of an anatomical region of a subject, generate first image data by rendering the three-dimensional image data using a first rendering method, generate second image data by rendering the three-dimensional image data using a second rendering method, adjust the second image data based on depth information and generate combined image data based on the first image data and the adjusted second image data.

A medical imaging system 10 according to an embodiment is illustrated schematically in FIG. 1. In the present embodiment, the system 10 comprises an ultrasound imaging system 12. In other embodiments, the system may comprise a computer tomography (CT) system, X-ray imaging system or other medical imaging system.

In the present embodiment, the ultrasound imaging system 12 is configured to acquire ultrasound data from an ultrasound scan and to process the ultrasound data to obtain an ultrasound image. The ultrasound imaging system 12 comprises a measurement probe 14. Any suitable type of ultrasound imaging system 12 and measurement probe 14 may be used.

Ultrasound images are formed by sending pulses of high frequency sound waves into an anatomical region of a subject from the measurement probe 14. These pulses echo off tissue in the anatomical region with different reflection properties and are returned to and detected at the measurement probe 14. The ultrasound imaging system 12 is configured to use the measurement of these reflected pulses to construct an image. In some embodiments, the subject is a patient.

The measurement probe 14 may be tilted to capture ultrasound data at different orientations or it may be able to capture three-dimensional (3D) ultrasound data directly.

For example, 3D ultrasound data can be obtained by specifically designing the measurement probe 14 for collecting the 3D data. Alternatively, 3D ultrasound data may be obtained by collecting a plurality of ultrasound images obtained by moving the measurement probe 14. For example, the measurement probe 14 may be tilted, with reflected pulses being captured at different orientations of the measurement probe 14. These reflected pulses captured at different orientations of the probe are processed to produce a three-dimensional array comprising a plurality of voxels representing the imaged anatomical region.

The 3D ultrasound data comprises an array of voxels, in which the value associated with each voxel is indicative of the presence of different types of tissue or substance. The 3D ultrasound data may also be referred to as 3D image data, ultrasound data, volumetric data, volumetric image data or a volume.

After capturing the 3D image data, a two-dimensional (2D) image is produced from a selected angle by applying a volume rendering technique to the 3D image data. The 2D image that is produced may comprise a helpful visualisation of the raw information captured by the ultrasound imaging system 12.

The ultrasound imaging system 12 comprises a main display screen 16 for displaying a main ultrasound image. The ultrasound machine 12 further comprises a scanner console 20. The scanner console 20 comprises a control screen 18 for displaying control information and input devices comprising various control knobs 19. The input devices may further comprise a computer keyboard, a mouse or a trackball (not shown). The control knobs 19 and/or other input devices may be used to adjust values for a plurality of hardware and software parameters. In the present embodiment, the control screen 18 is a touch screen, which is both a display device and a user input device. Further embodiments may comprise a control screen 18, display screen or main display screen 16 that does not form part of the ultrasound imaging system 12.

The ultrasound imaging system 12 comprises a data store 21. The ultrasound imaging system 12 comprises a processing apparatus 22 for processing of data, including 3D image data. The processing apparatus 22 comprises a Central Processing Unit (CPU) and Graphical Processing Unit (GPU). The processing apparatus 22 may also be referred to as a medical image processing apparatus.

The processing apparatus 22 includes processing circuitry 24. The processing circuitry 24 may be implemented in the CPU, in the GPU, or in a combination of the CPU and the GPU.

In the present embodiment, the processing apparatus 22 comprises rendering circuitry 26 configured to generate the 2D image from the 3D image data. The generated 2D image is a 2D projection of the 3D image data. The rendering circuitry 26 may in addition to the 3D image data, receive as an input, one or more parameters enabling it to provide the 2D image. The parameters include the position and orientation of a camera relative to a volume represented by the 3D image data. The 2D image is created from the perspective of this camera. For example, a position of the camera relative to the 3D image data may define a viewing direction of the anatomical region represented by the 2D image. The parameters may additionally include lighting information for providing illumination in the 2D image. The camera may also be referred to as a virtual camera. The camera may comprise a perspective camera, an autographic camera, a fish eye camera or any other suitable camera.

The rendering circuitry 26 is configured to use a rendering method or rendering algorithm. For example, the rendering method may comprise a volume rendering method, such as a direct volume rendering method. In direct volume rendering, the processing circuitry 24, e.g. the rendering circuitry 26, is configured to generate a ray for each desired image pixel. The rendering circuitry 26 may use a camera model. Each ray starts at the camera, e.g. a projection centre, and passes through an associated image pixel in an imaginary image plane between the camera and the 3D image data. Each ray is sampled at a plurality of points in the 3D image data. The points may be at equi-spaced intervals along the ray. At each point, the rendering circuitry 26 is configured to determine a sample based on the 3D image data, e.g. using an interpolation method, such as a trilinear interpolation method or the like. The rendering circuitry 26 is configured to apply a transfer function to the samples, e.g. to assign to each sample properties of colour and opacity. For example, the transfer function may map a value of each sample to a colour and opacity. Colour can be expressed as a colour value comprising values for a plurality of colour channels. Opacity may be expressed as an opacity value.

In some embodiments, the rendering method may comprise a surface volume rendering (SVR) method, such as global illumination rendering method or any other suitable rendering method.

The processing apparatus 22 comprising image processing circuitry 27. The image processing circuitry 27 is configured to blend image data using a blending method or blending mode.

The processing apparatus 22 comprises display circuitry 28 configured to display the 2D image or an image sequence, which comprises the 2D image, to a user on the display screen 16.

In the present embodiment, the circuitries 24, 26, 27, 28 are each implemented in the CPU and/or GPU by means of a computer program having computer-readable instructions that are executable to perform one or more operations of the medical image processing apparatus 10, the processing apparatus 22 and/or a medical image processing method of an embodiment described herein. In other embodiments, the circuitries may be implemented as one or more ASICs (application specific integrated circuits) or FPGAs (field programmable gate arrays).

In the present embodiment, the processing circuitry 24 is implemented in the CPU and/or GPU of processing apparatus 22 by means of a computer program having computer-readable instructions that are executable to perform one or more operations of the system 10. However, in other embodiments the processing circuitry may be implemented in software, hardware or any suitable combination of hardware and software. In some embodiments, the various circuitries may be implemented as one or more ASICs (application specific integrated circuits) or FPGAs (field programmable gate arrays).

The processing apparatus 22 also includes a hard drive and other components including RAM, ROM, a data bus, an operating system including various device drivers, and hardware devices including a graphics card. Such components are not shown in FIG. 1 for clarity.

FIGS. 2 and 3 show each a flow chart illustrating in overview a process according to an embodiment.

At stage 30, the processing circuitry 24 is configured to acquire 3D image data of an anatomical region of a subject. For example, the processing circuitry 24 may be configured to acquire the 3D image data from an ultrasound scan. Alternatively, the processing circuitry 24 may be configured to acquire the 3D image data from the data store 21. This stage has been omitted in FIG. 3 for sake of clarity.

At stage 32, the processing circuitry 24, e.g. the rendering circuitry 26, is configured to generate first image data and first depth information by rendering the 3D image data using a first rendering method. The first rendering method comprises a rendering method for visualising a surface of the anatomical region of the subject. The first rendering method comprises a global illumination rendering method, such as a first global illumination rendering method. This first global illumination rendering method may also be referred to as an opaque or fully opaque rendering method or an opaque render. It may be used for visualising a surface of an anatomical region of the subject. For example, it may be used to determine structural and/or spatial, such as 3D spatial information of the volume. It will be appreciated that in other embodiments, the first rendering method may comprise a surface rendering method or any other suitable rendering method.

The first global illumination rendering method may add lighting effects to a 3D image such that a subject of the image appears to be illuminated from a given position and/or direction. For example, a lighting model may be used that includes both direct illumination by light coming directly from a light source and indirect illumination, for example illumination by light that has been scattered from another surface.

In some embodiment, in the first global illumination rendering method, the first image data is rendered from a 3D image data set using a two-pass method in which a first pass creates a light volume, and a second pass uses the light volume to render the first image data for display.

The first pass may comprise a traversal from the light source into the 3D image data, in which virtual light is cast into the 3D image data. The irradiance due to the light source may be determined at each of a large array of points in the 3D image data using absorptive properties assigned to the voxels in dependence on the voxel values. The irradiance values at the array of points may be stored as a light volume. The light volume may be stored in data store or any other suitable memory of the processing apparatus 22. The light volume may be independent of the viewpoint.

A second pass may comprise a traversal through the light volume from a virtual camera, using the light volume to provide global lighting information. Rays may be cast from the camera, and irradiances from points along each ray may be integrated to provide pixel colour values for the first image data.

In the first global illumination rendering method, each point in the 3D image data is assigned a property that may be referred to as a reflection colour, an extinction colour, absorption colour, and/or attenuation colour. For example, the extinction colour may be expressed as an RGB colour. The reflection colour may be expressed as an opacity. The first global illumination rendering method may use two transfer functions that map a value of each sample to a colour and opacity. A first transfer function may determine the extinction colour. A second transfer function may determine a reflection colour. It will be appreciated that in some embodiments, the first global illumination rendering method may use only one transfer function to map each value of each sample to the opacity.

The first global illumination rendering method may require the use of more parameters than are used in the direct volume rendering method described above. Parameters used in the first global illumination rendering method may include, for example, one or more of a light position, a colour map, a brightness and one or more radiosity parameters.

The first image data may also be referred to as first rendered image data. The first image data comprises a plurality of first pixels. The first pixels may also be referred to as “a. pixels.”

FIG. 3 shows a first 2D image 42, which represents the first image data. It can be seen from FIG. 3 that in this embodiment, the anatomical region comprises an organ of the subject. In this embodiment, the organ comprises a uterus, including a foetus, of the subject. The anatomical region may comprise an anatomical structure or object.

The first depth information may take the form of a first depth map. The first depth map may indicate for each first pixel in the first image data a first depth or first distance to a sample in the 3D image data having a first predetermined opacity value. For example, the first depth or first distance may be understood as a depth or distance that a ray associated with each first pixel travels before it encounters the sample having the predetermined opacity value. Each first pixel may be associated with a first depth.

A direction of the first depth may comprise a direction of each ray from the camera to the 3D image data. For example, the direction of the first depth may comprise the direction of each ray from the camera through the associated first pixel in an imaginary image plane between the camera and the 3D image data. This direction may also be referred to as viewing direction. The direction of the first depth can be understood as the viewing direction of the first rendering method.

The first predetermined opacity value may define a first opacity threshold. For example, as described above, at each point in the 3D image data, the rendering circuitry 26 may be configured to compare each sample to the transfer function, e.g. the second transfer function used in the first rendering method. The rendering circuitry 26 may also be configured to accumulate the opacity values of the samples through the 3D image data. A distance or depth at which the accumulated opacity value reaches the first opacity threshold may be understood as the first depth or distance. The first depth may correspond to a surface depth of the anatomical region identified in the first rendering method. The terms “first depth” and “surface depth” may be interchangeably used. The first opacity threshold may define an amount of samples, e.g. along a ray, that are included in the first depth information. For example, when the opacity threshold decreases, a number of samples that are included in the first depth information may decrease. When the opacity threshold increases, a number of samples that are included in the first depth information may increase. The term “number of samples” may be understood as a number of samples for which the first opacity threshold is not met. An exemplary first depth map 44 is shown in FIG. 3.

At stage 34, the processing circuitry 24, e.g. the rendering circuitry 26, is configured to generate second image data and second depth information by rendering the three-dimensional image data using a second rendering method. The second image data comprises a plurality of second pixels. The second pixels may also be referred to as “b. pixels.” The first image data and/or the second image data may also be referred to as a plurality of pieces of rendered image data.

The second rendering method comprises at least one of a global illumination rendering method and a surface rendering method. The second rendering method is configured to visualise an internal structure of the anatomical region of the subject, e.g. without the use of a segmentation method. For example, the second rendering method is configured to show one or more surfaces of the anatomical region in a glass-like appearance. The one or more surfaces may comprise one or more internal and/or one or more external surfaces. The one or more surfaces may be shown as transparent shells. The second rendering method may also be referred to as a transparent render or a transparent rendering method or a ShadowGlass method.

As described above, the rendering circuitry 26 is configured to apply at least one transfer function to the samples, e.g. to assign to each sample properties of colour and opacity. In the following description, this transfer function will be referred to as a first transfer function. In the second rendering method, the rendering circuitry 26 is configured to adjust the first transfer function based on a second transfer function. The second transfer function maps one or more gradient values to one or more respective opacity values. The rendering circuitry 26 is configured to determine a gradient value associated with each sample. For example, the rendering circuitry 26 is configured to determine sample values of one or more adjacent samples in each direction in the 3D image data, e.g. in each of a x-direction, y-direction and z-direction. The adjacent samples may be understood as samples adjacent to the currently sampled sample. The rendering circuitry 26 is configured to determine the sample values of the adjacent samples to determine a change of the value of the current sample in each direction. The change of the value of the current sample is also referred to as a gradient. The gradient may be expressed as a vector. The rendering circuitry 26 is configured to determine a norm or length of the vector. The norm of the vector in each direction associated with each sample may be expressed as the second transfer function. The rendering circuitry 26 is configured to adjust the first transfer function by multiplying the opacity values of the first transfer function with the second transfer function.

The adjusted first transfer function may eliminate one or more opacity values for one or more samples, which comprise a small gradient in the x-direction, y-direction and z-direction, and visualise only samples with an increased gradient in the x-direction, y-direction and z-direction, e.g. one or more outer shells or surfaces of the anatomical region. As an opacity value is reduced in the second rendering method, e.g. via the multiplication of the opacity value of the first transfer function with the second transfer function, single shells or surfaces may remain transparent and opacity values, which are still accumulated along each rays, are reduced compared to an opaque rendering method, e.g. the first rendering method, even when a ray passes through multiple shells or surfaces in succession. For example, this can be seen in the head area of the foetus in a second 2D image 46, which is shown in FIG. 3.

In this embodiment, the second rendering method comprises a second global illumination rendering method. The second global illumination rendering method may require the use of more parameters than those used in the direct volume rendering method described above. Parameters used in the second global illumination rendering method may include, for example, one or more of a light position, a light direction, a light colour, a bidirectional reflectance distribution function (BRDF) parameter or setting, e.g. a bidirectional scattering distribution function (BSDF) parameter or setting. The bidirectional scattering distribution function may define how the way light reflects of a surface changes. The parameters may also be referred to as independent settings.

FIG. 3 shows the second 2D image 46, which represents the second image data.

The second depth information may take the form of a second depth map 48. The second depth map may indicate for each second pixel in the second image data a second depth or second distance to a sample in the 3D image data having a second predetermined opacity value. For example, the second depth or second distance may be understood as a depth or distance a ray associated with each second pixel travels before it encounters a sample having the predetermined opacity value. The second predetermined opacity value may be different from the first predetermined opacity.

The second predetermined opacity value may define a second opacity threshold. For example, as described above, at each point in the 3D image data, the rendering circuitry 26 may be configured to compare each sample to the transfer function, e.g. the first adjusted transfer function. The rendering circuitry 26 may also be configured to accumulate the opacity values of the samples through the 3D image data. A distance or depth at which the accumulated opacity value reaches the second opacity threshold may be understood as the second depth or distance. Each second pixel may be associated with a second depth. The second depth map may indicate a position of one or more surfaces of the anatomical region in the second image data. The second opacity threshold may define an amount of samples, e.g. along a ray, that are included in the second depth information. For example, when the opacity threshold decreases, a number of samples that are included in the second depth information may decrease. When the opacity threshold increases, a number of samples that are included in the second depth information may increase. The term “number of samples” may be understood as a number of samples for which the second opacity threshold is not met. An exemplary second depth map 48 is shown in FIG. 3.

A direction of the second depth may comprise a direction of each ray from the camera through the associated second pixel in an imaginary image plane between the camera and the 3D image data. For example, the direction of the second depth can be understood as a viewing direction of the second rendering method.

In some embodiments, the processing circuitry 24 is configured to normalise the first and second depth information, e.g. into a shared space. The shared space may also be referred to as a shared depth space. In the shared space, a depth of zero represents a closest point of a first pixel and/or a second pixel to the camera. A depth of one represents a furthest point of a first pixel and/or a second pixel to the camera. The normalisation of the first and second depth information may allow for a determination of a position of the first depth of each first pixel relative to a position of the second depth of each corresponding second pixel and/or a determination of a position of the second depth of each second pixel relative to a designated depth, which will be described in more detail below. The normalisation of the first and second depth information may allow for a depth of the buffer region to be used with different sets of 3D data, e.g. with requiring little or no modification. The depth of the buffer region represents a positive or a negative fraction of a whole depth in the shared space. The depth of the buffer region may also be referred to as a [0,1] depth value. The designated depth may also be referred to as a selected depth and these terms may be interchangeably used.

In some embodiments, the first and second depth information is not normalised. For example, the processing circuitry 24 may be configured to generate the first and second depth information using a same camera setting, e.g. the same camera angle, and a same 3D image data. In such embodiments, the processing circuitry 24 is configured to compare the first and second depth information with each other, e.g. to determine a position of the first depth of each first pixel relative to a position of the second depth of each corresponding second pixel and/or a determination of a position of the second depth of each second pixel relative to the designated depth.

The normalisation or comparison of the x-direction, y-direction, z-direction is shown at stage 35 of FIG. 2.

At stage 36, the processing circuitry 24 is configured to adjust the second image data based on the first and second depth information. This stage will be described below in more detail.

At stage 38, the processing circuitry is configured to generate combined image data representing the anatomical region of the subject. The processing circuitry 24 is configured to generate the combined image data based on the first image data and the adjusted second image data. This stage will also be described below in more detail.

At stage 40, the processing circuitry 24, e.g. the display circuitry 28, is configured to display the combined image data. FIG. 3 shows a 2D image 50, which represents the combined image data.

The processing circuitry 24 may be configured to repeat one or more of stages 32 to 40. For example, when a different view of the anatomical region is desired, the processing circuitry 24, e.g. the rendering circuitry 26, may be configured to render the 3D image data using a different position of the camera.

In a first iteration of the process, the processing circuitry 24, e.g. the rendering circuitry 26, is configured to render the three-dimensional image data using the first rendering method and to render the three-dimensional image data using the second rendering method, as described in stages 32 and 34 respectively. For example, stages 32 and 34 may be performed in parallel or sequentially.

In one or more subsequent iterations, the processing circuitry 24 is configured to alternately perform stages 32 and 34. For example, the processing circuitry 24, e.g. the rendering circuitry 26, is configured to alternately render the three-dimensional image data using at least one of the first and second rendering methods. This generates at least one of updated first depth information and updated second depth information and at least one of updated first image data and updated second image data. The updated first depth information, the updated second depth information, the updated first image data and the updated second image data may be generated in the same manner as that described in relation to stages 32 and 34. However, in each subsequent iteration only one of the updated first and second depth information and only one of the updated first and second image data is generated. This may allow for rendering of different views, where the changes between the views are small, with an increased performance.

For example, in response to generating the updated second image data, the processing circuitry 24 is configured to adjust the updated second image data based on previously generated first depth information and the updated second depth information. It will be appreciated that the term “previously” may refer to first depth information that has been generated in an iteration that was performed before a current iteration. The updated second image data may be adjusted as described in relation to stages 36 and 36a to 36e.

For example, in response to generating the updated first image data, the processing circuitry 24 is configured to adjust the previously generated second image data based on the updated first depth information and the previously generated second depth information. It will be appreciated that the term “previously” may refer to second depth information that has been generated in an iteration that was performed before a current iteration. The previously generated second image data may be adjusted as described in relation to stages 36 and 36a to 36e.

The processing circuitry 24 is configured to generate the combined image data based on at least one of: the previously generated first image data and the adjusted previously generated second image data and at least one other of the updated first image data and the adjusted updated second image data. For example, when the 3D image data is rendered using the first rendering method, e.g. stage 32 is performed, the processing circuitry 24 is configured to generate the combined image data based on the updated first image data and the adjusted previously generated second image data. It will be appreciated that the term “previously” may refer to second image data that has been adjusted in an iteration that was performed before a current iteration. The previously generated second image data may be adjusted as described in relation to stages 36 and 36a to 36e.

When the 3D image data is rendered using the second rendering method, e.g. stage 34 is performed, the processing circuitry 24 is configured to generate the combined image data based on the previously generated first image data and the adjusted updated second image data. It will be appreciated that the term “previously” may refer to first image data that has been generated in an iteration that was performed before a current iteration. The combined image data may be generated as described in relation to stages 38 and 38a to 38c.

FIG. 4 is a flow chart illustrating in overview further stages of the process shown in FIGS. 2 and 3.

At stage 36a, the processing circuitry 24 is configured to apply a first factor to each second pixel to adjust a value of at least one channel of each second pixel. For example, the processing circuitry 24 is configured to apply the first factor to each second pixel to adjust a maximum value of at least one channel of each second pixel. This may allow a user control over an overall intensity of a contribution of the second pixels to the combined image data.

The first factor may be an input parameter into the process. For example, the first factor may be adjustable and/or specified by a user.

Each second pixel may comprise a colour channel and an opacity channel. In the present embodiment, the processing circuitry 24 is configured to apply the first factor to a maximum value of the opacity channel of the second pixel. The terms “value of the opacity channel” may be interchangeably used with the terms “opacity value”. The opacity value of each pixel may be normalised to be between 0 and 1. Any of the changes to the opacity value of each second pixel described herein may be between 0 and 1.

The processing circuitry 24 is configured to decrease the maximum opacity value of the second pixel, e.g. to a predetermined maximum opacity value. The first factor may comprise a dimming factor. The term “dimming” may be understood as decreasing an opacity value, e.g. between 0 and 1. The dimming factor may also be referred to as a [0,1] dimming factor. The following description refers to changes to the opacity value of the second pixel. However, it will be appreciated that a colour value of the second pixel may additionally or alternatively be changed or adjusted.

In this embodiment, the processing circuitry 24 may be configured to multiply the first factor into the opacity channel of each second pixel. Each second pixel comprises an adjusted opacity value.

In some embodiments, the processing circuitry 24 is configured to maintain the adjusted opacity value of the second pixel as constant or unchanged in the buffer region. For example, the opacity value of the second pixel may be the same as the opacity value mentioned in stage 36a. For example, each second pixel in the buffer region can continue to contribute at its adjusted maximum opacity value. In such embodiments, the depth of the buffer region is positive.

In other embodiments, the processing circuitry 24 is configured to vary the adjusted opacity value of the second pixel in the buffer region. For example, the opacity value of the second pixel may decrease as will be described below. In such other embodiments, the depth of the buffer region is negative.

At stage 36b, the processing circuitry 24 is configured to determine or designate the designated depth. The processing circuitry 24 is configured to determine or designate the designated depth based on the first depth information. The designated depth will be referred to in the following as fadeout depth. The terms “designated depth” and “fadeout depth” may be interchangeably used.

For example, the processing circuitry 24 is configured to determine or designate the fadeout depth based on the first depth. As described above, the first depth may correspond to a depth of a surface of the anatomical region identified in the first rendering method. The first depth may also be referred to as a surface depth of the anatomical region. The fadeout depth is designated or determined in the viewing direction of the first rendering method. The processing circuitry 24 is further configured to determine or designate the fadeout depth based on the depth of the buffer region.

The processing circuitry 24 is configured to determine the depth of the buffer region. The buffer region is adjacent to the fadeout depth. The depth of the buffer region may be determined relative to the fadeout depth. The depth of the buffer region may also be referred to as buffer depth.

The processing circuitry 24 may be configured to determine the depth of the buffer region based on one or more properties of the anatomical region. For example, the processing circuitry 24 may be configured to automatically determine the depth of the buffer region based on the one or more properties of the anatomical region. The one or more properties of the anatomical region may comprise a dimension or size of the anatomical region and/or a thickness of the anatomical region. In embodiments where the anatomical region comprises a foetus, the one or more properties of the anatomical region comprise an age of the foetus. The age of the foetus may infer a size and/or thickness of the anatomical region, e.g. the foetus.

In some embodiments, the depth of the buffer region corresponds to a thickness of the anatomical region. The buffer region may also be referred to as a buffer zone. The depth of the buffer region may also be referred to as an optimal depth.

The fadeout depth is adjustable. For example, the processing circuitry 24 may be configured to vary or adjust the fadeout depth, e.g. by adjusting or varying the first opacity threshold, which was described above, and/or the depth of the buffer region.

In some embodiments, the depth of the buffer region may be an input parameter into the process. The depth of the buffer region may be adjustable, specified and/or determined by the user. This may allow for the fadeout depth to be adjusted by the user.

At stage 36c, the processing circuitry 24 is configured to determine for each second pixel whether a second depth associated with the second pixel is in front of the fadeout depth. For example, the processing circuitry 24 is configured to determine whether the second depth associated with the second pixel is smaller than the fadeout depth. The processing circuitry 24 is configured to make this determination based on the normalised first and second depth information or the comparison between the first and second depth information.

In response to a determination that the second depth associated with a second pixel is in front of the fadeout depth, the processing circuitry 24 is configured to maintain the opacity value, e.g. the adjusted opacity value, of the second pixel as constant or unchanged.

In response to the determination that the second depth associated with the second pixel is in front of the fadeout depth, the processing circuitry 24, e.g. the image processing circuitry 27, is configured to blend the second pixel with a corresponding first pixel, e.g. to generate a blended pixel. The combined image data comprises the blended pixel. The processing circuitry 24 e.g. the image processing circuitry 27, is configured to use a blend mode to blend the second pixel with the corresponding first pixel. The blend mode may be configured to retain and/or select a maximum opacity value of one of the first and second pixels. For example, a product of opacity values of the corresponding first and second pixels may be subtracted from a sum of the opacity values of the corresponding first and second pixels. In this embodiment, the blend mode comprises a lighten blend mode or a lighten only blend mode. However, it will be appreciated that in other embodiments, the blend mode may comprise a different blend mode, such as a multiply blend mode, an addition blend mode, a darken blend mode, an overlay blend mode, an “in blend mode”, an “atop blend mode” or any other suitable blend mode. This step may be part of stage 38 shown in FIG. 2. As such, this step is labelled as stage 38a in FIG. 3. The terms “blend mode” used herein may be interchangeably used with the terms “blending method.”

At stages 36d and 36e, the processing circuitry 24 is configured to change an opacity value of a second pixel associated with a second depth behind the fadeout depth.

For example, at stage 36d, in response to a determination that the second depth associated with a second pixel is not in front of the fadeout depth, the processing circuitry 24 is configured to determine a second factor for changing the opacity value of the second pixel. For example, the processing circuitry 24 may determine that the second depth associated with the second pixel is behind the fadeout depth and/or that the second depth associated with the second pixel is larger than the fadeout depth. The second factor may also be referred to as a fadeout factor or a [0,1] fadeout factor.

The second factor is determined based on the depth of the buffer region, the first and second depth information and a further gradient. For example, the second factor may be determined based on the further gradient and a relative depth between the fadeout depth and the second depth associated with the second pixel. For example, the fadeout depth, which may be based on the first depth and/or the depth of the buffer region, may be compared to the second depth associated with the second pixel to determine the relative depth. The second factor may be associated with the second pixel. The second factor may define a fadeout percentage, which is based on the further gradient and the relative depth. For example, at the fadeout depth, a value of the second factor is one. The value of the second factor decreases, e.g. gradually decreases, with increasing relative depth. At a relative depth where the second factor has eliminated a contribution of the second pixels, the second factor is zero.

For example, the second factor can be calculated as follows:

$${f}_{2} = 1 + G \times {d}_{r}$$

where f₂ is the second factor, G is the further gradient and dᵣ is the relative depth. It will be appreciated that in other embodiments, the second factor may be calculated differently. For example, in such other embodiments, the second factor may be additionally based on the first factor.

In some example, the second factor may be negative. In such example, the processing circuitry 24 is configured to restrict or clamp a value of the second factor to between one and zero. Additionally, the processing circuitry 24 is configured to set any negative values of the second factor as zero.

The further gradient may define a change of the opacity values of the second pixels with increasing relative depth. The further gradient comprises a linear gradient or a non-linear gradient. For example, the non-linear gradient may comprise one or more polynomial factors. The further gradient may be an input parameter into the process. The input parameter may be adjustable and/or specified by a user. For example, an input parameter of zero may represent no gradient. The input parameter may define a size of the gradient. For example, when the input parameter is a negative number, an increase of the negative number may cause an increase in the further gradient. The further gradient may also be referred to as fadeout slope or [−inf, 0] slope value.

At stage 36e, the processing circuitry 24 is configured to change an opacity value of the second pixel. For example, the processing circuitry 24, is configured to decrease the opacity value of the second pixel. This may generate an adjusted second pixel.

The processing circuitry 24 is configured to change, e.g. decrease, the opacity value according to the second factor. For example, the processing circuitry 24 is configured to multiply the second factor into the opacity channel of the second pixel.

At stage 38b, the processing circuitry 24, e.g. the image processing circuitry 27, is configured to blend the adjusted second pixel, which was generated at stage 36e, with a corresponding first pixel. The processing circuitry 24, e.g. the image processing circuitry 27, is configured to use a blend mode to blend the adjusted second pixel with the corresponding first pixel. This may generate a blended pixel. The blend mode may be configured to retain and/or select a maximum opacity value of one of the first pixel and the adjusted second pixel, as described above in relation to stage 38a. For example, the blend mode comprises a lighten blend mode, a lighten only blend mode or any other suitable blend mode. This step may also be part of stage 38 shown in FIG. 2.

At stage 38c, the processing circuitry 24 is configured to generate an interpolated pixel. The interpolated pixel is based on the blended pixel, which was generated at stage 38b, and a corresponding first pixel. The corresponding first pixel may be the same as the first pixel mentioned in relation to stage 38b.

The combined image data described in relation to stage 38 comprises the interpolated pixel. For example, each pixel of the combined image data corresponds to an interpolated pixel, which was generated at stage 38c or a blended pixel, which was generated at stage 38a.

In this embodiment, the processing circuitry 24 is configured to use a linear interpolation method to generate the interpolated pixel. The processing circuitry 24 is configured to apply the second factor as an interpolation factor to the blended pixel, e.g. prior to generating the interpolated pixel. It will be appreciated that in other embodiments, the processing circuitry may be configured to use a different interpolation method and/or interpolation factor.

It will be appreciated that one or more of stages 36a to 36e and stages 38a to 38c may be repeated for each second pixel of the second image data. The process described above may allow for a depth-aware blend of the first and second image data.

FIG. 5A illustrates an exemplary graph of an opacity value of the second pixels in dependence on the second depth. In the example shown in FIG. 5A, the first and second depth information have been normalised into a shared depth space.

It can be seen that the opacity value of each second pixel associated with a second depth in front of the fadeout depth is constant. As described above, the first factor is applied to each second pixel. This may result in the opacity value of each second pixel associated with a second depth in front of the fadeout depth has been adjusted to a maximum opacity value. The first factor comprises the dimming factor. As such, a zone in front of the fadeout depth may also be referred to as a dimming zone.

The opacity value of each second pixel associated with a second depth behind the fadeout depth gradually decreases with an increasing second depth. In this embodiment, the opacity value decreases in accordance with the second factor, which is linear. However, it will be appreciated that in other embodiments, the opacity value may decrease in accordance with a different function or factor, such as an inverse exponential function, a polynomial factor, a logarithmically decreasing function or any other suitable function or factor. As described above, the second factor comprises the fadeout factor. As such, a zone behind the fadeout depth may also be referred to as a fadeout zone.

In the example shown in FIG. 5A, the buffer region extends between the first depth and the fadeout depth. The buffer region is considered as a positive buffer region. For example, the depth of the buffer region comprises a positive value. However, it will be appreciated that in other embodiments, a depth of the buffer region may be zero or negative.

FIG. 5B illustrates another exemplary graph of an opacity value of the second pixels in dependence on the second depth. The graph shown in FIG. 5B is similar to the graph shown in FIG. 5A. As such, any features described in relation to the graph shown in FIG. 5A may also apply to the graph shown in FIG. 5B. Only differences will be described in the following.

In the example shown in FIG. 5B, a depth of buffer region is zero. As such, the fadeout depth is the same as the first depth.

FIG. 5C illustrates another exemplary graph of an opacity value of the second pixels in dependence on the second depth. The graph shown in FIG. 5C is similar to the graph shown in FIG. 5A. As such, any features described in relation to the graph shown in FIG. 5A may also apply to the graph shown in FIG. 5C. Only differences will be described in the following.

In the example shown in FIG. 5C, the buffer region extends between the first depth and the fadeout depth. However, in this example, the buffer region is considered as a negative buffer. For example, the depth of the buffer region comprises a negative value. As such, the opacity value of each second pixel associated with a second depth behind the fadeout depth starts to decrease in front of the first depth.

As described above, a user may adjust the depth of the buffer region to adjust the fadeout depth. For example, the user may adjust the depth of the buffer region to adjust a transition from the dimming zone to the fadeout zone around the first depth.

FIG. 6 is a flow chart illustrating in overview a process according to another embodiment.

At stage 60, the processing circuitry 24 is configured to acquire 3D image data of an anatomical region of a subject. Any features described above in relation to stage 30 may also apply to stage 60.

At stage 62, the processing circuitry 24, e.g. the rendering circuitry 26, is configured to generate first image data by rendering the 3D image data using the first rendering method. In this embodiment, the processing circuitry 24, e.g. the rendering circuitry 26, is also configured to generate depth information by rendering the 3D image data using the first rendering method. The depth information described in this embodiment may comprise the first depth information, as described above. The first image data comprises a plurality of first pixels.

As described above, the depth information may take the form of a depth map. The depth map may indicate for each first pixel in the first image data a first depth or first distance to a sample in the 3D image data having a predetermined opacity value. For example, the first depth or first distance may be understood as a depth or distance a ray associated with each first pixel travels before it encounters the sample having the predetermined opacity value. As described above, the predetermined opacity value may define an opacity threshold. For example, at each point in the 3D image data, the rendering circuitry 26 may be configured to compare each sample to the transfer function, e.g. the second transfer function used in the first rendering method. The rendering circuitry 26 may also be configured to accumulate the opacity values of the samples through the 3D image data. A distance or depth at which the accumulated opacity value reaches the opacity threshold may be understood as the first depth or distance. The depth may correspond to a depth of a surface of the anatomical region identified in the first rendering method. The opacity threshold may define an amount of samples, e.g. along a ray, that are included in the depth information. For example, when the opacity threshold decreases, a number of samples that are included in the depth information may decrease. When the opacity threshold increases, a number of samples that are included in the depth information may increase. The term “number of samples” may be understood as a number of samples for which the opacity threshold is not met. Each first pixel may be associated with a respective first depth.

At stage 64, the processing circuitry 24, e.g. the rendering circuitry 26, is configured to render the three-dimensional image data based on the depth information using the second rendering method to generate second image data. In this embodiment, no second depth information is generated, when rendering the 3D image data using the second rendering method. However, the processing circuitry 24 is configured to compare the first depth information to a current depth of each sample at a point along the ray during the second rendering method. This may allow a ray to be terminated early, e.g. when a relative depth indicates that the opacity value should be faded out, e.g. should be zero. This may allow the process to be performed with an increased speed and/or performance. Additionally or alternatively, a blending of the first and second image data may be improved.

In contrast, in a transparent rendering method where a ray is not terminated early during the rendering method, the ray may travel through the entirety of the 3D image data, e.g. due to the accumulated opacity values of the transparent surfaces or shells remaining low. For example, the ray may only be terminated, when it reaches another predetermined opacity threshold, which may be set for early termination and is different from the opacity threshold mentioned above, or when the ray reaches a back of the 3D image data. This may result in a decreased speed or performance of the second rendering method.

In this embodiment, the second image data comprises a plurality of second pixels. Stage 64 will be described in more detail below.

At stage 66, the processing circuitry is configured to generate combined image data representing the anatomical region of the subject. The processing circuitry 24 is configured to generate the combined image data based on the first image data and the second image data. The processing circuitry 24 is configured to blend the first image data with the second image data using a blend mode. For example, each first pixel is blended with a corresponding second pixel. In this embodiment, the blend mode comprises a lighten blend modus or a lighten only blend mode. However, it will be appreciated that in other embodiments, the blend mode may comprise a different blend mode, such as a multiply blend mode, an addition blend mode, a darken blend mode, an overlay blend mode or any other suitable blend mode.

At stage 68, the processing circuitry 24, e.g. the display circuitry 28, is configured to display the combined image data.

The processing circuitry 24 may be configured to repeat one or more of stages 62 to 68. For example, when a different view of the anatomical region is desired, the processing circuitry 24, e.g. the rendering circuitry 26, may be configured to render the 3D image data using a different position of the camera.

In a first iteration of the process, the processing circuitry 24, e.g. the rendering circuitry 26, is configured to render the three-dimensional image data using the first rendering method and to render the three-dimensional image data using the second rendering method, as described in stages 62 and 64 respectively.

In one or more subsequent iterations, the processing circuitry 24 is configured to alternately perform stages 62 and 64. For example, the processing circuitry 24 is configured to alternately render the three-dimensional image data using at least one of the first and second rendering methods. This generates at least one of updated depth information, updated first image data and updated second image data. The updated depth information, the updated first image data and the updated second image data may be generated in the same manner as that described in relation of stages 62 and 64. However, in each subsequent iteration, either the updated depth information and the updated first image data or the updated second image data is generated. This may allow for rendering of different views, where the changes between the views are small, with an increased performance.

The processing circuitry 24, e.g. the rendering circuitry 26, is configured to generate the updated second image data using previously generated depth information. It will be appreciated that the term “previously” may refer to depth information that has been generated in an iteration that was performed before a current iteration.

The processing circuitry 24 is configured to generate the combined image data based on at least one of the previously generated first image data and the previously generated second image data and at least one other of updated first image data and updated second image data.

For example, when the 3D image data is rendered using the first rendering method, the processing circuitry 24 is configured to generate the combined image data based on the updated first image data and the previously generated second image data. It will be appreciated that the term “previously” may refer to second image data that has been generated in an iteration that was performed before a current iteration.

When the 3D image data is rendered using the second rendering method, the processing circuitry 24 is configured to generate the combined image data based on the previously generated first image data and the updated second image data. It will be appreciated that the term “previously” may refer to first image data that has been generated in an iteration that was performed before a current iteration. The combined image data may be generated as described in relation to stage 66.

FIG. 7 is a flow chart illustrating in overview further stages of the process shown in FIG. 6. Stages 64a to 64 h may be performed as part of the second rendering method and stage 64.

The second rendering method comprises the volume rendering method described above. The processing circuitry 24, e.g. the rendering circuitry 26, is configured to generate a ray for each desired second pixel. For each ray, the processing circuitry 24, e.g. the rendering circuitry 26, is configured to determine a sample at a plurality of points in the 3D image data. The processing circuitry 24, e.g. the rendering circuitry 26, is configured to successively determine a plurality of samples at the plurality of points. For example, the processing circuitry 24, e.g. the rendering circuitry 26, may be configured to determine a sample at a point in the 3D image data before moving to a next point in the 3D image data to determine the next sample. As described above, at each point, the processing circuitry 24, e.g. the rendering circuitry 26, is configured to determine a sample based on the 3D image data, e.g. using an interpolation method, such as a trilinear interpolation method or the like. The processing circuitry 24, e.g. the rendering circuitry 26, is configured to apply a transfer function, e.g. the first adjusted transfer function, to the samples, e.g. to assign to each sample properties of opacity. The transfer function may also assign to each sample properties of colour.

At stage 64a, the processing circuitry 24, e.g. the rendering circuitry 26, is configured to apply a first factor to a colour value and/or opacity value assigned to the sample at each point. The processing circuitry 24, e.g. the rendering circuitry 26, is configured to apply the first factor to adjust the colour value and/or opacity value assigned to each sample. For example, the processing circuitry 24, e.g. the rendering circuitry 26, is configured to apply the first factor to adjust a maximum value of the colour value and/or opacity value assigned to each sample. The processing circuitry 24, e.g. the rendering circuitry 26, is configured to apply the first factor to decrease the maximum value of the colour value and/or opacity value assigned to each sample, e.g. to a predetermined maximum colour value and/or opacity value. The first factor comprises a dimming factor.

In this embodiment, the first factor is multiplied with an opacity value assigned to each sample. It will be appreciated that in other embodiments, the colour value assigned to each sample may be additionally or alternatively multiplied with the first factor.

At stage 64b, the processing circuitry 24 is configured to determine or designate a fadeout depth based on the depth information, which was generated at stage 62 shown in FIG. 6, and/or the depth of the buffer region described above.

At stage 64c, the processing circuitry 24, e.g. the rendering circuitry 26, is configured determine at each of the plurality of points whether a respective sample is determined in front of the fadeout depth. For example, the processing circuitry 24, e.g. the rendering circuitry 26, is configured to determine at each of the plurality of points whether a depth of the respective sample in the 3D image data is smaller than the fadeout depth.

The processing circuitry 24, e.g. the rendering circuitry 26, is configured to maintain at least one of the colour value and opacity value assigned to a sample that is determined in front of the designated depth as constant or unchanged.

In response to a determination that the sample is determined in front of the fadeout depth, the processing circuitry 24, e.g. the rendering circuitry 26, is configured to move to the next point, at stage 64d. For example, the processing circuitry 24, e.g. the rendering circuitry 26, is configured to determine whether a sample at a subsequent point of the plurality of points is determined in front of the fadeout depth.

The processing circuitry 24, e.g. the rendering circuitry 26, is configured to change at least one of the colour value and opacity value assigned to a sample that is determined behind the designated depth. For example, in response to a determination that the sample is not determined in front of the designated depth, the processing circuitry, e.g. the rendering circuitry 26, is configured to change the at least one of the colour value and opacity value assigned to the sample. This will be described in relation to stages 64e to 64h below.

For example, in response to a determination that the sample is not determined in front of the fadeout depth, the processing circuitry 24 is configured to determine a second factor, at stage 64e. For example, the processing circuitry 24, e.g. the rendering circuitry 26, may determine that the sample is determined behind the fadeout depth and/or a depth of the sample in the 3D image data is larger than the fadeout depth.

The processing circuitry 24 is configured to determine the second factor based on a further gradient and a relative depth between the fadeout depth and the current depth at a current point, e.g. the depth associated with the sample. For example, the processing circuitry 24 may be configured to compare the fadeout depth, which may be based on the first depth and/or the depth of the buffer region, to the current depth to determine the relative depth. This may result in the second factor being associated with the sample, e.g. the current sample. The second factor may define a fadeout percentage, which is based on the further gradient and the relative depth. For example, the second factor can be calculated as follows:

$${f}_{2} = 1 + G \times {d}_{r}$$

where f₂ is the second factor, G is the further gradient and dᵣ is the relative depth. It will be appreciated that in other embodiments, the second factor may be calculated differently. For example, in such other embodiments, the second factor may be additionally based on the first factor.

In some example, the second factor may be negative. In such example, the processing circuitry 24 is configured to restrict or clamp a value of the second factor to between one and zero. Additionally, the processing circuitry 24 is configured to set any negative values of the second factor as zero.

At stage 64f, the processing circuitry 24, e.g. the rendering circuitry 26, is configured to apply the second factor to the colour value and/or the opacity value assigned to the sample, e.g. to decrease the colour value and/or the opacity value assigned to the sample.

The second factor comprises a fadeout factor. The application of the second factor to the colour value and/or the opacity value assigned to each sample behind the fadeout depth may result in a decrease of the colour value and/or the opacity value with increasing depth of the ray in the 3D image volume.

In this embodiment, the second factor is multiplied with the opacity value assigned to the sample. For example, the further gradient may be an input parameter into the second rendering method. The input parameter may be adjustable and/or specified by a user, as described above. For example, processing circuitry 24, e.g. the rendering circuitry 26, is configured to determine the second factor based on the further gradient and the relative depth, as described above. The second factor may be used to fade out an opacity value assigned to the samples during the second rendering method, e.g. as the ray travels from the camera through the 3D image data. It will be appreciated that in other embodiments, the colour value assigned to the sample may be additionally or alternatively multiplied with the second factor.

The first factor and the buffer depth may also be input parameters into the second rendering method. For example, processing circuitry 24, e.g. the rendering circuitry 26, is configured to apply the first factor to a colour value and/or opacity value assigned to the sample at each point, as described above in relation to stage 64a. The processing circuitry 24 is configured to determine or designate the fadeout depth based on the depth information and the depth of the buffer region, as described above.

At stage 64g, the processing circuitry 24 is configured to determine whether the second factor is equal to zero. The value of the second factor decreases, e.g. gradually decreases, with increasing depth in the 3D image data to zero. For example, at the fadeout depth, a value of the second factor is one. At a termination depth, a value of the second factor is zero.

In response to a determination that the second factor is equal to zero, the processing circuitry 24, e.g. rendering circuitry 26, is configured to terminate a sample determination at stage 64h. For example, the processing circuitry 24, e.g. the rendering circuitry 26, is configured to terminate the rendering of the 3D image data using the second rendering method at the termination depth. This may allow for an early termination of the ray, which in turn may improve a speed of the second rendering method. By applying the second factor to the opacity value assigned to the sample, as described in relation to stage 64f, the opacity value assigned to the sample is decreased to zero at the termination depth.

In response to a determination that the second factor is not equal to zero, the processing circuitry 24, e.g. the rendering circuitry 26, is configured to move to the next point, at stage 64d. For example, the processing circuitry 24, e.g. the rendering circuitry 26, may be configured to determine that the second factor is larger than zero.

It will be appreciated that one or more of stages 64a to 64 h may repeated for each point of the plurality of points and/or for each second pixel of the second image data.

It will also be appreciated that features described above in relation to FIGS. 1 to 5 may also apply to stages 60 to 68 and/or stages 64a to 64h.

FIG. 8A illustrates an exemplary graph of an opacity value assigned to a number of samples in dependence on a depth in the 3D image data.

In can be seen that the opacity value assigned to each sample determined in front of the fadeout depth is constant. As described above, the first factor was applied to the opacity value assigned to each sample. This may result in the opacity value assigned to each sample determined in front of the fadeout depth comprising a maximum opacity value. As described above, the first factor comprises the dimming factor. As such, a zone in front of the fadeout depth may also be referred to as a dimming zone.

The opacity value assigned to each sample determined behind the fadeout depth decreases with increasing depth in the 3D image data. In this embodiment, the opacity value decreases in accordance with the second factor, which is linear. However, it will be appreciated that in other embodiments, the opacity value may decrease in accordance with a different function or factor, such as an inverse exponential function, a logarithmically decreasing function, a polynomial factor or any other suitable function or factor.

As described above, the second factor comprises the fadeout factor. As such, a zone behind the fadeout depth may also be referred to as a fadeout zone. At the termination depth, the second factor is zero, as described above. This in turn may result in an opacity value assigned to a sample determined at the termination depth being zero.

In the example shown in FIG. 8A, the buffer region extends between the first depth and the fadeout depth. As can be seen in FIG. 8A, the opacity value assigned to each sample determined in front of the fadeout depth is constant. The buffer region is considered as a positive buffer region. For example, the depth of the buffer region comprises a positive value. A depth of the buffer region may be determined as described above. However, it will be appreciated that in other embodiments, a depth of the buffer region may be zero or negative.

FIG. 8B illustrates another exemplary graph of an opacity value assigned to a number of samples in dependence on a depth in the 3D image data. The graph shown in FIG. 8B is similar to the graph shown in FIG. 8A. As such, any features described in relation to the graph shown in FIG. 8A may also apply to the graph shown in FIG. 8B. Only differences will be described in the following.

In the example shown in FIG. 8B, a depth of the buffer region is zero. As such, the fadeout depth is the same as the first depth.

FIG. 8C illustrates another exemplary graph of an opacity value assigned to a number of samples in dependence on a depth in the 3D image data. The graph shown in FIG. 8C is similar to the graph shown in FIG. 8A. As such, any features described in relation to the graph shown in FIG. 8A may also apply to the graph shown in FIG. 8C. Only differences will be described in the following.

In the example shown in FIG. 8C, the buffer region extends between the first depth and the fadeout depth. However, in this example, the buffer region is considered as a negative buffer. For example, the depth of the buffer region comprises a negative value. As such, the opacity value of each sample determined behind the fadeout depth starts to decrease in front of the first depth.

As described above, a user may specify and/or adjust a depth of the buffer region to adjust the fadeout depth. For example, the user may adjust the depth of the buffer region to adjust a transition from the dimming zone to the fadeout zone around the first depth.

FIG. 9A shows a 2D image representing the first image data and FIG. 9B shows a 2D image representing the second image data. It can be seen from FIG. 9B that a definition of the edges of the foetus and the surrounding area is improved, e.g. compared to the 2D image shown in FIG. 9A.

FIG. 9C shows a 2D image representing a blend of the first and second image data shown in FIGS. 9A and 9B, respectively. The 2D image shown in FIG. 9C was generated by blending the first and second image data using a lighten blend mode. It can be seen that there are a number of artefacts 52 in the 2D image representing the blend of the first and second image data. The artefacts 52 may be due to structures and/or noise 54 in the second image data, which are indicated in FIG. 9B. The artefacts 52 may degrade a quality of the blended 2D image and/or introduce noise.

FIG. 9D shows a 2D image that has been generated using the process described above in relation to FIGS. 2 to 5C. It can be seen that the number of artefacts is reduced compared to FIG. 9C. In addition, a definition of edges of the foetus and the surrounding area is improved. By decreasing the opacity value of each second pixel associated with a second depth behind the fadeout depth, the number of artefacts and/or noise may be reduced. This may result in a contribution of the second image data being reduced or faded out depending on how far is behind the fadeout depth. This in turn may lead to an improved quality of the generated image and/or a cleaner generated image.

FIG. 10A shows another exemplary 2D image representing first image data. In this example, the anatomical region comprises the head and neck of the foetus. A part of the face of the foetus is difficult to see in this 2D image due to it being in a shaded area.

FIG. 10B shows another exemplary 2D image representing first image data. In this example, a position of the light source used in the first rendering method is changed compared to a position of the light source used to generate the 2D image shown in FIG. 10A. As can be seen in FIG. 10B, face of the foetus is harder to see than that shown in FIG. 10A.

FIG. 10C shows another exemplary 2D image representing the second image data.

FIG. 10D shows a 2D image representing a blend of the first and second image data shown in FIGS. 10A and 10C. The 2D image shown in FIG. 10D was generated by blending the first and second image data shown in FIGS. 10A and 10C using a lighten blend mode. It can be seen that the blending of the first and second image data has resulted in improvements of the visibility of the face of the foetus compared to the 2D image shown in FIG. 10A.

FIG. 10E shows a 2D image representing a blend of the first and second image data shown in FIGS. 10B and 10C. The 2D image shown in FIG. 10E was generated by blending the first and second image data shown in FIGS. 10B and 10C using a lighten blend mode. In FIG. 10E, some of the internal structures of the head of the foetus, which are visible in FIG. 10C, are also visible. Some or all of these internal structures 56 may be distracting and/or considered as artefacts in the 2D image shown in FIG. 10E.

FIG. 10F shows a 2D image that has been generated using the process described above in relation to FIGS. 2 to 5C. The 2D image shown in FIG. 10F has been generated based on the first and second image data represented by the 2D images shown in FIGS. 10A and 10C.

FIG. 10G shows a 2D image that has been generated using the process described in relation to FIGS. 2 to 5C described herein. The 2D image shown in FIG. 10G has been generated based on the first and second image data represented by the 2D images shown in FIGS. 10B and 10C.

In both FIGS. 10F and 10G, it can be seen that the number of artefacts and/or the noise is reduced compared to FIGS. 10D and 10E, respectively. For example, the internal structures 56 appear less dominant in FIG. 10 G. The processes described herein may allow for cleaner 2D images, e.g. with improved edge definition, to be generated. By decreasing the opacity value of each second pixel having a second depth behind the fadeout depth, the number of artefacts and/or noise may be reduced. This in turn may result in an improved image quality.

FIG. 11 shows a comparison of two 2D images that have been generated using the process described above in relation to FIGS. 2 to 5C. The two 2D images shown in FIG. 11 have been generated using a different buffer region. The 2D image shown on the left, which is labelled as image (A), has been generated using a positive value for the depth of the buffer region. The 2D image shown on the right, which is labelled as image (B), has been generated using a negative value for the depth of the buffer region. It can be seen from this comparison that the adjustment or selection of the depth of the buffer region can determine how far in front or behind the first depth, a contribution of the second pixels or samples in the second rendering method is decreased. This may allow for different structures of the anatomical region to be visualised. For example, it can be seen that there are nearly no internal structure of the foetus visible in the image labelled (B), whereas some internal structures are visible in the image labelled (A).

FIG. 11 also shows an exemplary display part 58 configured to display a number of input parameters of the process. The display part 58 may be part of the command display 18, which is shown in FIG. 1. However, it will be appreciated that in other embodiments, the display part may be part of another display of the medical imaging system.

In FIG. 11, the display part 58 on the left, which is labelled (C), is associated with the 2D image shown on the left of FIG. 11. The display part 58 on the right, which is labelled (D), is associated with the 2D image shown on the right of FIG. 11. In this embodiment, the input parameters include the first factor, the depth of the buffer region and the further gradient. The further gradient is labelled as “gradient” in FIG. 11. As described above, one or more of these input parameters may be adjustable, specified by the user and/or determined by the processing circuitry 24.

Each of the input parameters shown in FIG. 11 may be adjusted by means of a slider or slider bar 59, only one of which is indicated in FIG. 11 for the sake of clarity. In this embodiment, the values of the first factor and the further gradient, which were used to generate the 2D image illustrated in FIG. 11, are the same. However, it can be seen that a depth of the buffer region used to generate the 2D image on the left of FIG. 11 is positive and a depth of the buffer region used to generate the 2D image on the right of FIG. 11 is negative. For example, by adjusting the depth of the buffer region, the fadeout depth may be adjusted.

Certain embodiments provide a medical image processing method comprising acquiring three-dimensional image data, acquiring a depth map (or depth information) related to the three-dimensional image data, acquiring a plurality of pieces of rendered image data by performing a first rendering, for visualizing an internal structure of an object, of the three-dimensional image data based on the depth map and by performing a second rendering, for visualizing a surface of the object, of the three-dimensional image data, and combining the plurality of pieces of rendered image data.

Certain embodiments provide a medical image processing method comprising acquiring three-dimensional image data, acquiring a plurality of pieces of rendered image data by performing a first rendering, for visualizing an internal structure of an object, of the three-dimensional image data and by performing a second rendering, for visualizing a surface of the object, of the three-dimensional image data, acquiring a depth map (depth information) related to the three-dimensional image data, and combining the plurality of pieces of rendered image data based on the depth map.

The first rendering may be at least one of a volume rendering method and ShadowGlass method.

The second rendering may be at least one of a surface rendering method and Global Illumination (SVR) method.

The second rendering may be performed based on the depth map.

In front of a designated depth, an opacity of the first rendering may be constant.

Behind the designated depth, the opacity of the first rendering may be changed, e.g. the opacity gradually decreases.

The designated depth may be set based on the surface depth of the object identified by the second rendering.

The designated depth may be set using the surface depth of the object identified by the second rendering and a specified buffer. The designated depth may adjustable.

Certain embodiments provide a medical imaging method comprising:
a. one opaque render of ultrasound data with depth map,
b. one transparent render of the same volume with its own but separate depth map as well as other independent settings, such as BSDF, lights, etc.,
c. method for normalizing depth values of render a. and b. into a shared scene space,
d. method for lighten-blending a. and b. renders using their depth maps, which includes the following:
i. [0,1] dimming factor for dimming the maximum opacity of b. pixels when their relative depth is in front of the matching a. pixel;
ii. [0,1] depth value for creating a buffer zone behind a. pixels' depth where b. pixels can continue to contribute at maximum contribution l;
iii. [−inf,0] slope value for calculating a [0,1] fadeout factor of b. pixel opacity to blend behind depth of a. pixel and depth value ii;
iv. Lighten blend of a. pixels and their corresponding b. pixels after the latter had their opacity dimmed and faded out according to steps i.-iii;
V. Linear interpolation of the result of a. and the result from the lighten blend iv., where the factor for iv. in this interpolation is the [0,1] fadeout factor calculated in iii;

The lighten method mentioned in d. and iv. may be replaced by different blending methods, such as an over blending method, in blending method, atop blending method, darken blending method, add blending method, multiply blending method, etc.

The fadeout slope mentioned in iii. may be a non-linear fadeout slope described by different input parameters.

An optimal depth of the buffer zone mentioned in ii. may be automated.

Performance and image quality may be improved by using the depth map of a. as well as the fadeout parameters ii. and iii. in the original b. render to establish termination depths.

Performance may be improved by alternating between updating only image a. or image b. for each blending update.

The dimming and fading out may be applied to all channels of b. pixel rather than just the opacity channel.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

The disclosure includes one or more corresponding embodiments or features in isolation or in various combinations whether or not specifically stated (including claimed) in that combination or in isolation. It should be understood that features defined above in accordance with any embodiment of the present disclosure may be utilized, either alone or in combination with any other defined feature, in any other embodiment or to form a further aspect or embodiment of the disclosure.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical image processing apparatus comprising processing circuitry configured to:
acquire three-dimensional image data of an anatomical region of a subject;
generate first image data by rendering the three-dimensional image data using a first rendering method;
generate second image data by rendering the three-dimensional image data using a second rendering method and depth information; and
generate combined image data representing the anatomical region of the subject based on the first image data and the second image data.

2. The medical image processing apparatus of claim 1, wherein the depth information is generated using the first rendering method.

3. The medical image processing apparatus of claim 1, wherein when using the second rendering method, the processing circuitry is configured to assign at least one of a colour value and an opacity value to each of a plurality of samples corresponding to a plurality of points in the three-dimensional image data.

4. The medical image processing apparatus of claim 2, wherein the processing circuitry is configured to determine at each of the plurality of points whether a respective sample is determined in front of a designated depth designated in a viewing direction of the first rendering method.

5. The medical image processing apparatus of claim 4, wherein the processing circuitry is configured to maintain at least one of the colour value and opacity value assigned to a sample that is determined in front of the designated depth as constant or unchanged.

6. The medical image processing apparatus of claim 4, wherein the processing circuitry is configured to change at least one of the colour value and opacity value assigned to a sample that is determined behind the designated depth.

7. The medical image processing apparatus of claim 4, wherein the processing circuitry is configured to designate the designated depth based on a surface depth of the anatomical region, the surface depth being identified by rendering the three-dimensional image data using the first rendering method.

8. The medical image processing apparatus of claim 4, wherein the processing circuitry is configured to designate the designated depth based on a depth of a buffer region and a surface depth of the anatomical region, the surface depth being identified by rendering the three-dimensional image data using the first rendering method.

9. The medical image processing apparatus of claim 4, wherein the designated depth is adjustable.

10. The medical image processing apparatus of claim 1, wherein
the first rendering method comprises a rendering method for visualising a surface of the anatomical region of the subject and the second rendering method comprises a method for visualising an internal structure of the anatomical region of the subject.

11. The medical image processing apparatus of claim 1, wherein
the first rendering method comprises at least one of a surface rendering method and a global illumination rendering method; and
the second rendering method comprises at least one of a volume rendering method, a transparent rendering method and a ShadowGlass method.

12. The medical image processing apparatus of claim 8, wherein the processing circuitry is configured to at least one of:
determine the depth of a buffer region based on one or more properties of the anatomical region; and
maintain at least one of the colour value and the opacity value assigned to each of the plurality of samples as constant in the buffer region.

13. A medical image processing method comprising:
acquiring three-dimensional image data of an anatomical region of a subject;
generating first image data by rendering the three-dimensional image data using a first rendering method;
generating second image data by rendering the three-dimensional image data using a second rendering method and depth information; and
generating combined image data representing the anatomical region of the subject based on the first image data and the second image data.

14. A medical image processing apparatus comprising processing circuitry configured to:
acquire three-dimensional image data of an anatomical region of a subject;
generate first image data by rendering the three-dimensional image data using a first rendering method;
generate second image data by rendering the three-dimensional image data using a second rendering method;
adjust the second image data based on depth information; and
generate combined image data based on the first image data and the adjusted second image data.

15. The medical image processing apparatus of claim 14, wherein the second image data comprises a plurality of second pixels and the processing circuitry is configured to determine for each of the plurality of second pixels whether a second depth associated with a second pixel is in front of a designated depth designated in a viewing direction of the first rendering method.

16. The medical image processing apparatus of claim 14, wherein the processing circuitry is configured to maintain a value of at least one channel of a second pixel associated with a second depth in front of the designated depth as constant or unchanged.

17. The medical image processing apparatus of claim 14, wherein the processing circuitry is configured to change a value of at least one channel of a second pixel associated with a second depth behind the designated depth.

18. The medical image processing apparatus of claim 14, wherein the processing circuitry is configured to designate the designated depth based on a surface depth of the anatomical region, the surface depth being identified by rendering the three-dimensional image data using the first rendering method.

19. The medical image processing apparatus of claim 14, wherein the processing circuitry is configured to designate the designated depth based on a depth of a buffer region and a surface depth of the anatomical region, the surface depth being identified by rendering the three-dimensional image data using the first rendering method.

20. A medical image processing method comprising:
acquiring three-dimensional image data of an anatomical region of a subject;
generating first image data by rendering the three-dimensional image data using a first rendering method;
generating second image data by rendering the three-dimensional image data using a second rendering method;
adjusting the second image data based on depth information; and
generating combined image data based on the first image data and the adjusted second image data.
